(19) United States
(12) Patent Application Publication
(10) Pub. No.: US 2005/0271691 A1
(43) Pub. Date: Dec. 8, 2005

(54) Prepackaged aqueous pharmaceutical formulations

(75) Inventor: Mitchell S. Karl, Boca Raton, FL (US)

Correspondence Address: RUDEN, MCCLOSKY, SMITH, SCHUSTER & RUSSELL, P.A., 222 LAKEVIEW AVE, SUITE 800, WEST PALM BEACH, FL, 33401-6112, US

(73) Assignee: Cardio Combos LLC

(21) Appl. No.: 11/145,666

(22) Filed: Jun. 6, 2005

Related U.S. Application Data
(63) Continuation-in-part of application No. 10/785,169, filed on Feb. 23, 2004.
(60) Provisional application No. 60/449,470, filed on Feb. 21, 2003.

(51) Int. Cl. A61K 31/56; A61K 9/00

(52) U.S. Cl. 424/400

(57) ABSTRACT

Prepackaged aqueous pharmaceutical compositions for the treatment of a condition are disclosed including at least two different pharmacologically active agents for the treatment of the condition, a buffering agent to buffer the composition, and an osmotic-adjusting agent. Additionally, a method of forming a prepackaged aqueous pharmaceutical composition for the treatment of a condition is disclosed, the method including the steps of mixing at least two different pharmacologically active agents, adding a buffering agent to the mixed at least two different pharmacologically active agents, and adding an osmotic-adjusting agent to the mixed at least two different pharmacologically active agents.

### CROSS REFERENCE TO RELATED APPLICATION

The present application is a continuation-in-part of U.S. patent application Ser. No. 10/785,169, filed Feb. 23, 2004, which claims the priority of U.S. provisional patent application No. 60/449,470, filed Feb. 21, 2003.

### FIELD OF THE INVENTION

The invention relates generally to the fields of medicine and pharmacology. More particularly, the present invention relates to pharmaceutical formulations for oral drug delivery.

### BACKGROUND OF THE INVENTION

Conventional means for delivering biologically-active agents, including, but not limited to, pharmaceutical and therapeutic agents, to animals are often severely limited by chemical barriers and physical barriers imposed by the body. Oral delivery of many biologically-active agents would be the route of choice if not for chemical and physico-chemical barriers such as the extreme and varying pH in the gastro-intestinal (GI) tract, exposure to powerful digestive enzymes, and the impermeability of gastro-intestinal membranes to the active agent. Among the numerous agents that are not typically suitable for oral administration are biologically-active peptides such as calcitonin and insulin. Examples of other compounds that are affected by these physico-chemical barriers are polysaccharides and particularly mucopolysaccharides, including, but not limited to, heparin; heparinoids; antibiotics; and other organic substances. These agents are rapidly destroyed in the gastro-intestinal tract by acid hydrolysis, enzymes, or the like.

Prior methods for orally administering vulnerable pharmacological agents have relied on the co-administration of adjuvants (e.g., resorcinols and non-ionic surfactants such as polyoxyethylene oleyl ether and n-hexadecyl polyethylene ether) to increase artificially the permeability of the intestinal walls; and on the co-administration of enzymatic inhibitors (e.g., pancreatic trypsin inhibitor, diisopropylfluorophosphate (DFF) and trasylol) to inhibit enzymatic degradation. Liposomes have also been described as drug delivery systems for insulin and heparin. See, for instance, U.S. Pat. No. 4,239,754. However, broad spectrum use of the aforementioned drug delivery systems is precluded for reasons including: (1) the need to use toxic amounts of adjuvants or inhibitors; (2) the lack of suitable low MW cargoes; (3) the poor stability and inadequate shelf life of the systems; (4) the difficulties in manufacturing the systems; (5) the failure of the systems to protect the active ingredient; and (6) the failure of the systems to promote absorption of the active agent.

Thus, there is still a need in the art for simple and inexpensive oral delivery systems for drug compositions that are easily prepared and that can deliver a broad range of biologically-active agents.

### SUMMARY OF THE INVENTION

The present invention eliminates the above-mentioned needs for a simple and inexpensive oral delivery system for drug compositions by providing prepackaged aqueous pharmaceutical compositions for the treatment of conditions such as HIV/AIDS, cancer, and heart failure.

Accordingly, the invention features a prepackaged aqueous pharmaceutical composition for the treatment of a condition including at least two different pharmacologically active agents for the treatment of the condition, a buffering agent to buffer the composition, and an osmotic-adjusting agent. Conditions to be treated include HIV/AIDS and cancer. The at least two different pharmacologically active agents include anti-angiogenesis and anti-viral agents. Buffering agents include at least one of acetate, glutamate, citrate, tartrate, benzoate, lactate, gluconate, phosphate and glycine. Osmotic-adjusting agents include at least one of sodium chloride, dextrose, sodium bicarbonate, calcium chloride, potassium chloride, sodium lactate, Ringer's solution and lactated Ringer's solution.

In another aspect the invention features a method of forming a prepackaged aqueous pharmaceutical composition for the treatment of a condition. The method includes the steps of mixing at least two different pharmacologically active agents, adding a buffering agent to the mixed at least two different pharmacologically active agents, and adding an osmotic-adjusting agent to the mixed at least two different pharmacologically active agents.

In still another aspect the invention features a process for the administration of a prepackaged aqueous solution or dispersion of at least two different pharmacologically active agents for the treatment of a condition. The process includes selecting a predetermined dosage amount for each of said at least two different pharmacologically active agents, the dosage amounts selected based upon patient characteristics, mixing the dosage amounts with a buffering agent and an osmotic-adjusting agent, the pharmacologically active agents having stability with one another in aqueous solution, and providing the pharmacologically active agents having stability with one another in aqueous solution to a suitable patient for oral administration.

### DETAILED DESCRIPTION OF THE PREFERRED EMBODIMENT

Physicians acceptance of combination pill therapy has been limited by heretofor very restricted and limited dosing combinations that do not allow for patient factors that might necessitate dose adjustments. The inadequate available variations are viewed as limiting their utility in clinical practice. However, if this obstacle could be circumvented and if the physician had multiple options to customize combination pre-packaged therapy for the individual patient based on common but clinically important differences, then physicians would likely embrace the concept.

Patients frequently complain about the cost of each of their medications and look for substitutes for particularly costly ones in their regimen. Patents often run out of, forget to take some of, and have difficulty swallowing medications. Patients may question the validity of a multiple drug regime and/or feel overmedicated by the number of pills they must swallow.

These problems are addressed by a prepackaged combination of daily medication for a particular condition (e.g., HIV/AIDS, cancer, heart failure) in liquid form tailored to specific ranges for use by individual patients having differing characteristics and selected from a chart by the patient's physician.

For example, combo “A” might contain the most commonly prescribed dosage of a standard regime for congestive heart failure:

| | | |
|---|---|---|
| Digoxin/Lanoxin | 25 | mg |
| Lasix/Furosamide | 20 | mg |
| Potassium Chloride | 10 | mg |
| Altace/Rampiril | 5 | mg |

As another example, for those with conduction system disease, bradycardia or renal disease, Digoxin is omitted from the regimen in combo “A”-2. For those with low blood pressure the Altace dosage is reduced or eliminated. Greater diuretic dosages would be provided in the combination for those who have such a need.

Although not every patient's needs fit neatly and exactly into an offered combination, a combination that very closely fits the patient's needs could be selected as most suitable and could then modified by the addition of one or two pills to the regime. This would not therefore diminish the utilization of this concept as it could be applied in whole or in part to most patients.

In all cases, the physician would be able to select the desired combination and dosing for the patient; and the patient would enjoy the greater simplicity, decreased cost, easier compliance, and greater confidence associated with prepackaged daily dosing.

Packaging could take the form of a plastic disposable dispenser with twist-off cap. Medication could be in the form of a combined liquid/elixir. Nutraceuticals and vitamins may be included in the drug regime if sufficient evidence supports their use.

### EXAMPLES

Cardio Combo A—for Congestive Heart Failure

| | |
|---|---|
| A1 | Digoxin-.25 mg; Lasix-20 mg; KLL-10 meg; Altace-5 mg |
| A2 | Digoxin-.125 mg; Lasix-40 mg; KLL-10 meg; Altace-2.5 mg |
| A3 | Lasix-40 mg; KLL-20 mg; Altace-2.5; CQ10 |
| A4 | Digoxin-.125 mg; Lasix-20 mg; KLL-10 mg; CQ10 |
| A5 | Digoxin-.125 mg; Lasix-40 mg; CQ10 |
| A6 | Digoxin-.25 mg; Lasix-20 mg; KLL-10 mg; Altace-2.5 + |
| | Coreg-3.1 (a single 3.1 mg core pill to be taken later in |
| | the day). |
| A7 | Same as A6 with 6.25 mg coreg |
| A8 | Same as A6 with 12.5 mg coreg |
| A9 | Same as A6 with 25 mg coreg |

Cardio Combo B—for Coronary Artery Disease:

| | |
|---|---|
| B1 | ASA-81 mg; Statin; B complex, Vitamin E |
| B2 | Same as B1, but with greater Statin dose |
| B3 | Same as B2 with addition of niacin |
| B4 | Same as B1 plus nitrate |
| B5 | Same as B4 plus beta blocker |
| B6 | Same as B5 plus calcium channel blocker |

Although only a few exemplary embodiments of the present invention have been described in detail above, those skilled in the art will readily appreciate that numerous modifications are to the exemplary embodiments are possible without materially departing from the novel teachings and advantages of this invention. Accordingly, all such modifications are intended to be included within the scope of this invention as defined in the following claims.

1. A prepackaged aqueous pharmaceutical composition for the treatment of a condition comprising:
at least two different pharmacologically active agents for the treatment of the condition;
a buffering agent to buffer said composition; and
an osmotic-adjusting agent.

2. The prepackaged aqueous pharmaceutical composition of claim 1, wherein the condition is selected from the group consisting of HIV/AIDS and cancer.

3. The composition according to claim 1 wherein said at least two different pharmacologically active agents are selected from the group consisting of anti-angiogenesis and anti-viral agents.

4. The composition according to claim 1 wherein said buffering agent comprises at least one of acetate, glutamate, citrate, tartrate, benzoate, lactate, gluconate, phosphate and glycine.

5. The composition according to claim 1 wherein said osmotic-adjusting agent comprises at least one of sodium chloride, dextrose, sodium bicarbonate, calcium chloride, potassium chloride, sodium lactate, Ringer's solution and lactated Ringer's solution.

6. A method of forming a prepackaged aqueous pharmaceutical composition for the treatment of a condition, said method comprising the steps of:
mixing at least two different pharmacologically active agents;
adding a buffering agent to said mixed at least two different pharmacologically active agents; and
adding an osmotic-adjusting agent to said mixed at least two different pharmacologically active agents.

7. A process for the administration of a prepackaged aqueous solution or dispersion of at least two different pharmacologically active agents for the treatment of a condition, the process comprising:
selecting a predetermined dosage amount for each of said at least two different pharmacologically active agents, said dosage amounts selected based upon patient characteristics;
mixing said dosage amounts with a buffering agent and an osmotic-adjusting agent, said pharmacologically active agents having stability with one another in aqueous solution; and
providing said pharmacologically active agents having stability with one another in aqueous solution to a suitable patient for oral administration.
